(19) United States
(12) Patent Application Publication
(10) Pub. No.: US 2025/0002447 A1
(43) Pub. Date: Jan. 2, 2025

(54) METHOD FOR PREPARING 2-AMINO-5-CHLORO-N,3-DIMETHYLBENZAMIDE

(71) Applicant: Hubei Kecy Chemical Co., Ltd., Qianjiang (CN)

(72) Inventors: Haiping MU, Qianjiang (CN); Qiang LI, Qianjiang (CN); Tao YAN, Qianjiang (CN); Qingfang YANG, Qianjiang (CN); Qionghui YUAN, Qianjiang (CN); Xingzhong LU, Qianjiang (CN)

(21) Appl. No.: 18/595,197

(22) Filed: Mar. 4, 2024

(30) Foreign Application Priority Data
Jun. 20, 2023 (CN) 202310738265.0

(51) Int. Cl. C07C 231/02; C07C 231/12; C07C 249/02

(52) U.S. Cl. CPC C07C 231/02; C07C 231/12; C07C 249/02

(57) ABSTRACT

A method for preparing 2-amino-5-chloro-N,3-dimethylbenzamide, including: subjecting o-toluidine to acylation reaction with an acylation agent to obtain o-methylacetanilide; subjecting the o-methylacetanilide to chlorination reaction with a chlorination agent to obtain 4-chloro-2-methylacetanilide; subjecting the 4-chloro-2-methylacetanilide to oximation reaction with an oximation agent to obtain 4-chloro-2-(hydroxyimino)-N-(2-methylphenyl) acetamide; subjecting the 4-chloro-2-(hydroxyimino)-N-(2-methylphenyl) acetamide to cyclization reaction with sulfuric acid to obtain 5-chloro-7-methylisatin; subjecting the 5-chloro-7-methylisatin to oxidation reaction with an oxidation agent to obtain 5-chloro-7-methylisatoic anhydride; and subjecting the 5-chloro-7-methylisatoic anhydride to ammonolysis reaction with monomethylamine to obtain the 2-amino-5-chloro-N,3-dimethylbenzamide

### CROSS REFERENCE TO RELATED APPLICATION

This patent application claims the benefit and priority of Chinese Patent Application No. 202310738265.0 filed with the China National Intellectual Property Administration on Jun. 20, 2023, the disclosure of which is incorporated by reference herein in its entirety as part of the present application.

### TECHNICAL FIELD

The present disclosure relates to the technical field of chemical synthesis, and specifically relates to a method for preparing 2-amino-5-chloro-N,3-dimethylbenzamide.

### BACKGROUND

2-amino-5-chloro-N,3-dimethylbenzamide (K amine) is classified as a heterocyclic compound, and is an important intermediate for dyes, pesticides, and pharmaceuticals. K amine is widely used in the fields of pesticides, pharmaceuticals, and dyes, and has a promising market prospect. K amine is the most critical intermediate for chlorantraniliprole. Chlorantraniliprole is a very efficient lepidopteran insecticide developed by DuPont in the United States, and has been on the market in China since 2008. Chlorantraniliprole is the first choice for control of agricultural pests such as Pieris rapae, Ostrinia nubilalis, Grapholita molesta, and Helicoverpa assulta. Chlorantraniliprole is a broad-spectrum insecticide with a reliable and stable control effect even at a low dose, and pests stop feeding immediately after chlorantraniliprole is applied. After being applied, chlorantraniliprole has a relatively long-lasting effect and is not affected by rain flushing. Chlorantraniliprole can provide an immediate and long-term protective effect when applied at any growth stage of a crop. Chlorantraniliprole occupies an important position in the field of pesticides worldwide. K amine, as a key intermediate for chlorantraniliprole, has received close attention, and a synthesis technique of K amine has always been a research focus inside and outside China.

A first existing solution for synthesizing K amine is as follows: K amine is synthesized with o-toluidine as a raw material. However, a reaction in the first existing solution requires a large amount of sodium sulfate and leads to large amounts of gas, water, and residue wastes. A second existing solution for synthesizing K amine is as follows: K amine is synthesized with m-toluic acid as a raw material. However, a reaction in the second existing solution involves nitrification and high-pressure hydrogenation, resulting in a complicated process and an unsafe production process. Therefore, there is a need to develop a method for preparing K amine that involves a simple post-treatment, small amounts of gas, water, and residue wastes, lower pollution, and lower cost.

### SUMMARY

In view of the technical problems in the prior art, the present disclosure provides a method for preparing 2-amino-5-chloro-N,3-dimethylbenzamide that involves a simple post-treatment, small amounts of gas, water, and residue wastes, a low pollution, and a low cost.

In order to solve the above technical problems, the present disclosure provides a method for preparing 2-amino-5-chloro-N,3-dimethylbenzamide, including the following steps:
subjecting o-toluidine to acylation reaction with an acylation agent to obtain o-methylacetanilide, as shown in the following equation:

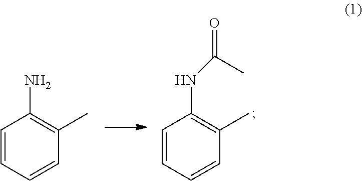

subjecting the o-methylacetanilide to chlorination reaction with a chlorination agent to obtain 4-chloro-2-methylacetanilide, as shown in the following equation:

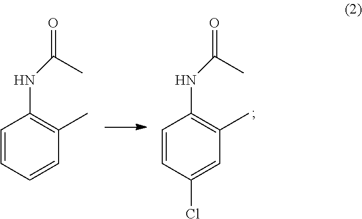

subjecting the 4-chloro-2-methylacetanilide to oximation reaction with an oximation agent to obtain 4-chloro-2-(hydroxyimino)-N-(2-methylphenyl) acetamide, as shown in the following equation:

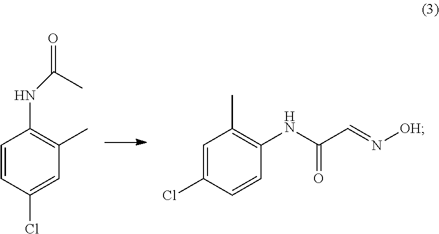

subjecting the 4-chloro-2-(hydroxyimino)-N-(2-methylphenyl) acetamide to cyclization reaction with sulfuric acid to obtain 5-chloro-7-methylisatin, as shown in the following equation:

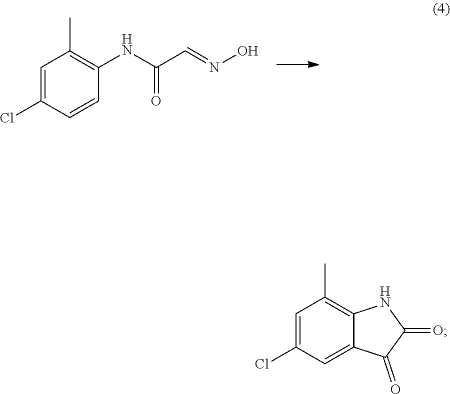

subjecting the 5-chloro-7-methylisatin to oxidation reaction with an oxidation agent to obtain 5-chloro-7-methylisatoic anhydride, as shown in the following equation:

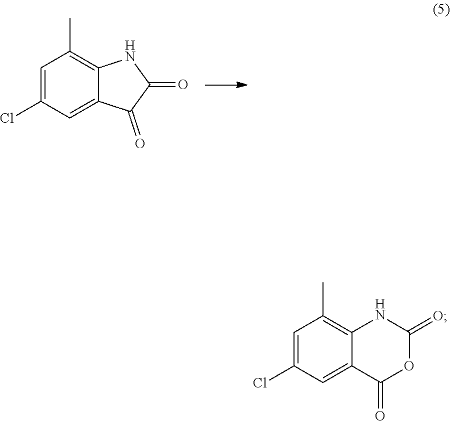

and
subjecting the 5-chloro-7-methylisatoic anhydride to ammonolysis reaction with monomethylamine to obtain the 2-amino-5-chloro-N,3-dimethylbenzamide, as shown in the following equation:

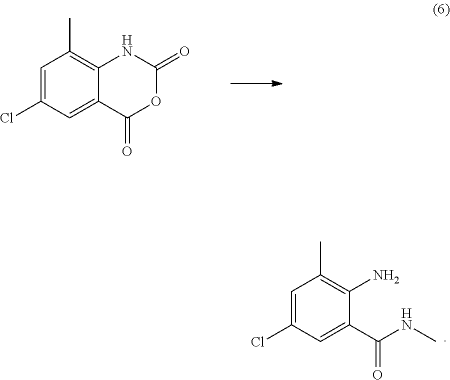

The following improvements may be further made to the present disclosure based on the above technical solution.

In some embodiments, the subjecting o-toluidine to acylation reaction with an acylation agent to obtain o-methylacetanilide specifically includes:

adding the acylation agent to a solution of the o-toluidine under stirring, and performing the acylation reaction at a first preset reaction temperature until the acylation reaction is completed, to obtain a first reaction system; and subjecting the first reaction system to cooling, suction filtration, water-washing, and oven-drying to obtain the o-methylacetanilide,

where a solvent in the solution of the o-toluidine is at least one selected from the group consisting of dichloromethane, dichloroethane, acetic acid, acetic anhydride, chlorobenzene, and xylene; the acylation agent is acetic acid or acetic anhydride; a molar ratio of the o-toluidine to the acylation agent is in a range of 1:0.9 to 1:1.5; a molar ratio of the o-toluidine to the solvent in the solution of the o-toluidine is in a range of 1:3 to 1:7; and the first preset reaction temperature is in a range of −10° C. to 135° C.

In some embodiments, the subjecting the o-methylacetanilide to chlorination reaction with a chlorination agent to obtain 4-chloro-2-methylacetanilide specifically includes:

adding the chlorination agent to a solution of the o-methylacetanilide under stirring, and performing the chlorination reaction at a second preset reaction temperature until the chlorination reaction is completed, to obtain a second reaction system; and subjecting the second reaction system to cooling, suction filtration, and oven-drying to obtain the 4-chloro-2-methylacetanilide,

where a solvent in the solution of the o-methylacetanilide is at least one selected from the group consisting of methanol, ethanol, dichloromethane, dichloroethane, acetic acid, chlorobenzene, and water; the chlorination agent is at least one selected from the group consisting of chlorine gas, N-chlorosuccinimide (NCS), hydrogen peroxide/hydrochloric acid, oxygen gas/hydrochloric acid, sulfuryl chloride, thionyl chloride, phosphorus trichloride, phosphorus pentachloride, and sodium hypochlorite; a molar ratio of the o-methylacetanilide to the chlorination agent is in a range of 1:1.1 to 1:1.5; a molar ratio of the o-methylacetanilide to the solvent in the solution of the o-methylacetanilide is in a range of 1:3 to 1:8; and the second preset reaction temperature is in a range of −10° C. to 105° C.

In some embodiments, the subjecting the 4-chloro-2-methylacetanilide to oximation reaction with an oximation agent to obtain 4-chloro-2-(hydroxyimino)-N-(2-methylphenyl) acetamide specifically includes:

adding the oximation agent to a solution of the 4-chloro-2-methylacetanilide under stirring, and performing the oximation reaction at a third preset reaction temperature until the oximation reaction is completed, to obtain a third reaction system; and subjecting the third reaction system to suction filtration, water-washing, and oven-drying to obtain the 4-chloro-2-(hydroxyimino)-N-(2-methylphenyl) acetamide,

where a solvent in the solution of the 4-chloro-2-methylacetanilide is selected from the group consisting of methanol, ethanol, dichloromethane, dichloroethane, acetic acid, chlorobenzene, and water; the oximation agent is selected from the group consisting of methyl nitrite, ethyl nitrite, n-butyl nitrite, isopropyl nitrite, isoamyl nitrite, and n-amyl nitrite; a molar ratio of the 4-chloro-2-methylacetanilide to the oximation agent is in a range of 1:1.1 to 1:2.0; a molar ratio of the 4-chloro-2-methylacetanilide to the solvent in the solution of the 4-chloro-2-methylacetanilide is in a range of 1:3 to 1:20; and the third preset reaction temperature is in a range of −10° C. to 115° C.

In some embodiments, the subjecting the 4-chloro-2-(hydroxyimino)-N-(2-methylphenyl) acetamide to cyclization reaction with sulfuric acid to obtain 5-chloro-7-methylisatin specifically includes:

adding the 4-chloro-2-(hydroxyimino)-N-(2-methylphenyl) acetamide to an aqueous solution of the sulfuric acid under stirring, and performing the cyclization reaction at a fourth preset reaction temperature until the cyclization reaction is completed, to obtain a fourth reaction system; and subjecting the fourth reaction system to cooling, suction filtration, and oven-drying to obtain the 5-chloro-7-methylisatin,

where a concentration of the aqueous solution of the sulfuric acid is in a range of 75 wt % to 98 wt %; a molar ratio of the 4-chloro-2-(hydroxyimino)-N-(2-methylphenyl) acetamide to the sulfuric acid is in a range of 1:3 to 1:30; and the fourth preset reaction temperature is in a range of 20° C. to 115° C.

In some embodiments, the subjecting the 5-chloro-7-methylisatin to oxidation reaction with an oxidation agent to obtain 5-chloro-7-methylisatoic anhydride specifically includes:

adding the oxidation agent dropwise to a solution of the 5-chloro-7-methylisatin under stirring, and performing the oxidation reaction at a fifth preset reaction temperature until the oxidation reaction is completed, to obtain a fifth reaction system; and subjecting the fifth reaction system to cooling, suction filtration, water-washing, and oven-drying to obtain the 5-chloro-7-methylisatoic anhydride,

where the oxidation agent is at least one selected from the group consisting of hydrogen peroxide, peroxyacetic acid, n-butanol peroxide, chromium trioxide, potassium permanganate, sodium hypochlorite, potassium chlorate, potassium peroxymonosulfate, and sodium persulfate; a molar ratio of the 5-chloro-7-methylisatin to the oxidation agent is in a range of 1:1.1 to 1:2.1; and the fifth preset reaction temperature is in a range of −10° C. to 110° C.

In some embodiments, the subjecting the 5-chloro-7-methylisatoic anhydride to ammonolysis reaction with monomethylamine to obtain the 2-amino-5-chloro-N,3-dimethylbenzamide specifically includes:

adding an aqueous solution of the monomethylamine dropwise to a solution of the 5-chloro-7-methylisatoic anhydride under stirring, and performing the ammonolysis reaction at a sixth preset reaction temperature until the ammonolysis reaction is completed, to obtain a sixth reaction system; and subjecting the sixth reaction system to suction filtration, water-washing, and oven-drying to obtain the 2-amino-5-chloro-N,3-dimethylbenzamide,

where a concentration of the aqueous solution of the monomethylamine is in a range of 10 wt % to 40 wt %; a solvent in the solution of the 5-chloro-7-methylisatoic anhydride is selected from the group consisting of methanol, ethanol, dichloromethane, dichloroethane, acetonitrile, ethyl acetate, and water; a molar ratio of the 5-chloro-7-methylisatoic anhydride to the monomethylamine is in a range of 1:1.1 to 1:3.1; and the sixth preset reaction temperature is in a range of −10° C. to 110° C.

The present disclosure has the following beneficial effects:

In the present disclosure, o-toluidine is subjected to acylation reaction with an acylation agent to obtain o-methylacetanilide; the o-methylacetanilide is subjected to chlorination reaction with a chlorination agent to obtain 4-chloro-2-methylacetanilide; the 4-chloro-2-methylacetanilide is subjected to oximation reaction with an oximation agent to obtain 4-chloro-2-(hydroxyimino)-N-(2-methylphenyl) acetamide; the 4-chloro-2-(hydroxyimino)-N-(2-methylphenyl) acetamide is subjected to cyclization reaction with sulfuric acid to obtain 5-chloro-7-methylisatin; the 5-chloro-7-methylisatin is subjected to oxidation reaction with an oxidation agent to obtain 5-chloro-7-methylisatoic anhydride; and the 5-chloro-7-methylisatoic anhydride is subjected to ammonolysis reaction with monomethylamine to obtain 2-amino-5-chloro-N,3-dimethylbenzamide. The method of the present disclosure involves mild conditions and simple and safe operations in each step, results in small amounts of gas, water, and residue wastes and little pollution, has a low cost and a high product purity, shows prominent social and economic benefits, and is suitable for large-scale industrial production.

### BRIEF DESCRIPTION OF THE DRAWINGS

One or more embodiments are exemplarily illustrated through corresponding accompanying drawings, and these exemplary descriptions do not constitute a limitation to the embodiments. Components with the same reference numerals in the accompanying drawings represent similar components, and the accompanying drawings are not limited by a scale unless otherwise specified.

FIG. 1 shows a flow chart of the method for preparing 2-amino-5-chloro-N,3-dimethylbenzamide according to an embodiment of the present disclosure.

FIG. 2 shows the synthesis process of 2-amino-5-chloro-N,3-dimethylbenzamide according to an embodiment of the present disclosure.

The implementation of the objects, the functional characteristics, and the advantages of the present disclosure will be further described below in conjunction with the embodiments and accompanying drawings.

### DETAILED DESCRIPTION OF THE EMBODIMENTS

The technical solutions of the embodiments of the present disclosure are clearly and completely described below in conjunction with the accompanying drawings in the embodiments of the present disclosure. Apparently, the described embodiments are merely some, rather than all, of the embodiments of the present disclosure. All other embodiments obtained by those of ordinary skill in the art based on the embodiments of the present disclosure without creative efforts shall fall within the scope of the present disclosure.

It should be noted that all the directional indications (such as upper, lower, left, right, front, and rear) in the embodiments of the present disclosure are used merely to explain relative position relationships, motion situations, or the like of components in specific gestures (as shown in the accompanying drawings). If the specific gestures change, the directional indications also change accordingly.

Moreover, the terms such as “first” and “second” described in the embodiments of the present disclosure are used herein only for the purpose of description and are not intended to indicate or imply relative importance, or implicitly indicate the number of indicated technical features. Therefore, features defined by “first” and “second” may explicitly or implicitly include at least one of the features. Further, the technical solutions of various embodiments may be combined with each other, but it must be on the basis that a combination thereof can be implemented by those of ordinary skill in the art. In case of a contradiction with the combination of the technical solutions or a failure to implement the combination, it should be considered that the combination of the technical solutions does not exist, and is not within the scope of the present disclosure.

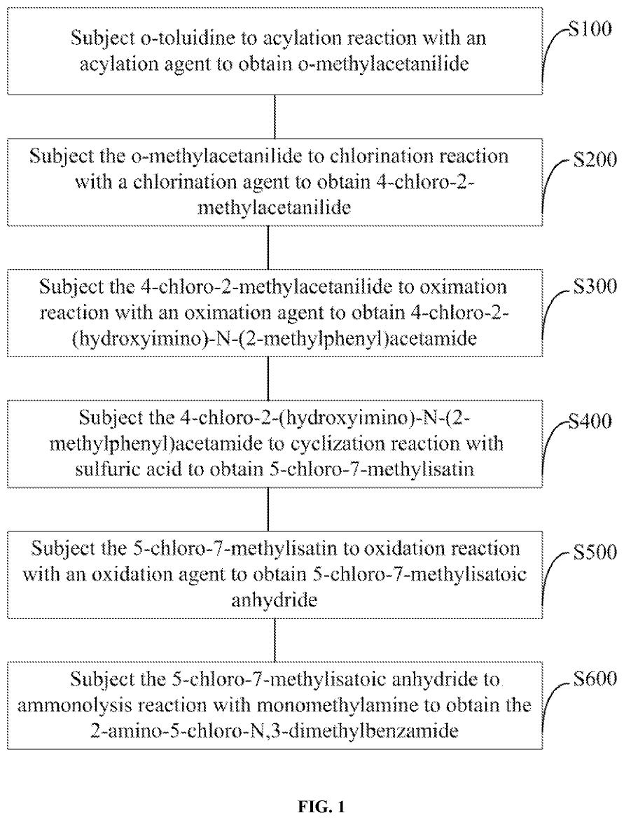

The present disclosure provides a method for preparing 2-amino-5-chloro-N,3-dimethylbenzamide, as shown in FIG. 1. The method for preparing 2-amino-5-chloro-N,3-dimethylbenzamide includes the following steps:

S100: o-Toluidine is subjected to acylation reaction with an acylation agent to obtain o-methylacetanilide, as shown in the following equation:

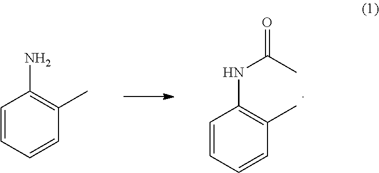

S100 specifically includes: the acylation agent is added to a solution of the o-toluidine under stirring, and the acylation reaction is performed at a first preset reaction temperature until the acylation reaction is completed, to obtain a first reaction system; and the first reaction system is subjected to cooling, suction filtration, water-washing, and oven-drying to obtain the o-methylacetanilide.

In some embodiments, a solvent in the solution of the o-toluidine is an organic solvent, and the organic solvent is at least one selected from the group consisting of dichloromethane, dichloroethane, acetic acid, acetic anhydride, chlorobenzene, and xylene.

In some embodiments, the acylation agent is acetic acid or acetic anhydride.

In some embodiments, a molar ratio of the o-toluidine to the acylation agent is in a range of 1:0.9 to 1:1.5. In some embodiments, the molar ratio of the o-toluidine to the acylation agent is 1:1.0, 1:1.1, 1:1.2, 1:1.3, or 1:1.4.

In some embodiments, a molar ratio of the o-toluidine to the solvent in the solution of the o-toluidine is in a range of 1:3 to 1:7. In some embodiments, the molar ratio of the o-toluidine to the solvent in the solution of the o-toluidine is 1:4, 1:5, or 1:6.

In some embodiments, the first preset reaction temperature is in a range of −10° C. to 135° C. In some embodiments, the first preset reaction temperature is −10° C., 0° C., 10° C., 20° C., 30° C., 40° C., 50° C., 60° C., 70° C., 80° C., 90° C., 100° C., 110° C., 120° C., or 130° C.

Specifically, a specified amount of the o-toluidine is fed into a reactor, an appropriate amount of the solvent is added to the reactor, and the acylation agent is added under stirring; the acylation reaction is performed at the first preset reaction temperature until the acylation reaction is completed, to obtain the first reaction system; and the first reaction system is cooled and centrifuged, and a resulting filter cake is washed with water until neutral and then oven-dried to obtain the o-methylacetanilide.

S200: the o-methylacetanilide is subjected to chlorination reaction with a chlorination agent to obtain 4-chloro-2-methylacetanilide, as shown in the following equation:

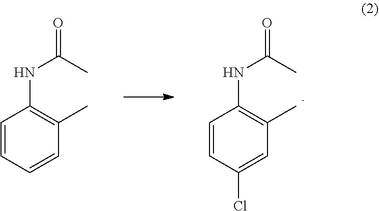

S200 specifically includes: the chlorination agent is added to a solution of the o-methylacetanilide under stirring, and the chlorination reaction is performed at a second preset reaction temperature until the chlorination reaction is completed, to obtain a second reaction system; and the second reaction system is subjected to cooling, suction filtration, and oven-drying to obtain the 4-chloro-2-methylacetanilide.

In some embodiments, a solvent in the solution of the o-methylacetanilide is at least one selected from the group consisting of methanol, ethanol, dichloromethane, dichloroethane, acetic acid, chlorobenzene, and water.

In some embodiments, the chlorination agent is at least one selected from the group consisting of chlorine gas, NCS, hydrogen peroxide/hydrochloric acid, oxygen gas/hydrochloric acid, sulfuryl chloride, thionyl chloride, phosphorus trichloride, phosphorus pentachloride, and sodium hypochlorite.

In some embodiments, a molar ratio of the o-methylacetanilide to the chlorination agent is in a range of 1:1.1 to 1:1.5. In some embodiments, the molar ratio of the o-methylacetanilide to the chlorination agent is 1:1.2, 1:1.3, or 1:1.4.

In some embodiments, a molar ratio of the o-methylacetanilide to the solvent in the solution of the o-methylacetanilide is in a range of 1:3 to 1:8. In some embodiments, the molar ratio of the o-methylacetanilide to the solvent in the solution of the o-methylacetanilide is 1:4, 1:5, 1:6, or 1:7.

In some embodiments, the second preset reaction temperature is in a range of −10° C. to 105° C. In some embodiments, the second preset reaction temperature is −10° C., 0° C., 10° C., 20° C., 30° C., 40° C., 50° C., 60° C., 70° C., 80° C., 90° C., or 100° C.

Specifically, an appropriate amount of the solvent is fed into a reactor, then the o-methylacetanilide obtained in the previous step is fed into the reactor, and the chlorination agent is added under stirring; the chlorination reaction is performed at the second preset reaction temperature until the chlorination reaction is completed, to obtain the second reaction system; and the second reaction system is cooled and centrifuged, and a resulting filter cake is oven-dried to obtain the 4-chloro-2-methylacetanilide.

S300: the 4-chloro-2-methylacetanilide is subjected to oximation reaction with an oximation agent to obtain 4-chloro-2-(hydroxyimino)-N-(2-methylphenyl) acetamide, as shown in the following equation:

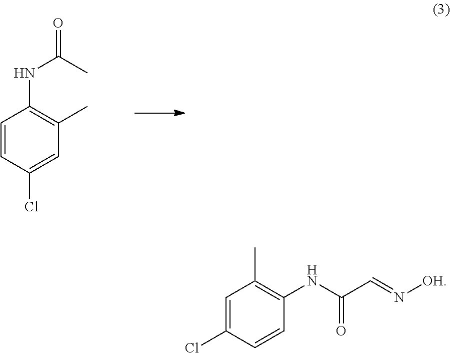

S300 specifically includes: the oximation agent is added to a solution of the 4-chloro-2-methylacetanilide under stirring, and the oximation reaction is performed at a third preset reaction temperature until the oximation reaction is completed, to obtain a third reaction system; and the third reaction system is subjected to suction filtration, water-washing, and oven-drying to obtain the 4-chloro-2-(hydroxyimino)-N-(2-methylphenyl) acetamide.

In some embodiments, a solvent in the solution of the 4-chloro-2-methylacetanilide is selected from the group consisting of methanol, ethanol, dichloromethane, dichloroethane, acetic acid, chlorobenzene, and water.

In some embodiments, the oximation agent is selected from the group consisting of methyl nitrite, ethyl nitrite, n-butyl nitrite, isopropyl nitrite, isoamyl nitrite, and n-amyl nitrite.

In some embodiments, a molar ratio of the 4-chloro-2-methylacetanilide to the oximation agent is in a range of 1:1.1 to 1:2.0. In some embodiments, the molar ratio of the 4-chloro-2-methylacetanilide to the oximation agent is 1:1.2, 1:1.3, 1:1.4, 1:1.5, 1:1.6, 1:1.7, 1:1.8, or 1:1.9.

In some embodiments, a molar ratio of the 4-chloro-2-methylacetanilide to the solvent is in a range of 1:3 to 1:20. In some embodiments, the molar ratio of the 4-chloro-2-methylacetanilide to the solvent is 1:4, 1:5, 1:6, 1:7, 1:8, 1:9, 1:10, 1:11, 1:12, 1:13, 1:14, 1:15, 1:16, 1:17, 1:18, or 1:19.

In some embodiments, the third preset reaction temperature is in a range of −10° C. to 115° C. In some embodiments, the third preset reaction temperature is −10° C., 0° C., 10° C., 20° C., 30° C., 40° C., 50° C., 60° C., 70° C., 80° C., 90° C., 100° C., or 110° C.

Specifically, a specified amount of the solvent is fed into a reactor, the 4-chloro-2-methylacetanilide obtained in the previous step is fed into the reactor, and the oximation agent is added under stirring; the oximation reaction is performed at the third preset reaction temperature until the oximation reaction is completed, to obtain the third reaction system; and the third reaction system is cooled and centrifuged, and a resulting filter cake is washed with water and then oven-dried to obtain the target product 4-chloro-2-(hydroxyimino)-N-(2-methylphenyl) acetamide.

S400: the 4-chloro-2-(hydroxyimino)-N-(2-methylphenyl) acetamide is subjected to cyclization reaction with sulfuric acid to obtain 5-chloro-7-methylisatin, as shown in the following equation:

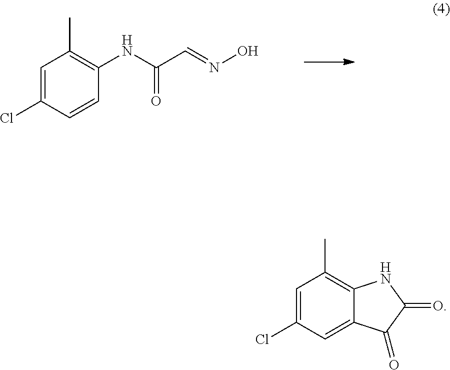

S400 specifically includes: the 4-chloro-2-(hydroxyimino)-N-(2-methylphenyl) acetamide is added to an aqueous solution of the sulfuric acid under stirring, and the cyclization reaction is performed at a fourth preset reaction temperature until the cyclization reaction is completed, to obtain the fourth reaction system; and the fourth reaction system is subjected to cooling, suction filtration, and oven-drying to obtain the 5-chloro-7-methylisatin.

In some embodiments, a concentration of the aqueous solution of the sulfuric acid is in a range of 75 wt % to 98 wt %. In some embodiments, the concentration of the aqueous solution of the sulfuric acid is 80 wt %, 85 wt %, 90 wt %, or 95 wt %.

In some embodiments, a molar ratio of the 4-chloro-2-(hydroxyimino)-N-(2-methylphenyl) acetamide to the sulfuric acid is in a range of 1:3 to 1:30. In some embodiments, the molar ratio of the 4-chloro-2-(hydroxyimino)-N-(2-methylphenyl) acetamide to the sulfuric acid is 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, or 1:29.

In some embodiments, the fourth preset reaction temperature is in a range of 20° C. to 115° C. In some embodiments, the fourth preset reaction temperature is 30° C., 40° C., 50° C., 60° C., 70° C., 80° C., 90° C., 100° C., or 110° C.

Specifically, a specified amount of the aqueous solution of the sulfuric acid is fed into a reactor, and the 4-chloro-2-(hydroxyimino)-N-(2-methylphenyl) acetamide obtained in the previous step is fed into the reactor under stirring; the cyclization reaction is performed at the fourth preset reaction temperature until the cyclization reaction is completed, to obtain the fourth reaction system; and the reaction system is cooled and centrifuged, and a resulting filter cake is washed with water and oven-dried to obtain the target product 5-chloro-7-methylisatin.

S500: the 5-chloro-7-methylisatin is subjected to oxidation reaction with an oxidation agent to obtain 5-chloro-7-methylisatoic anhydride, as shown in the following equation:

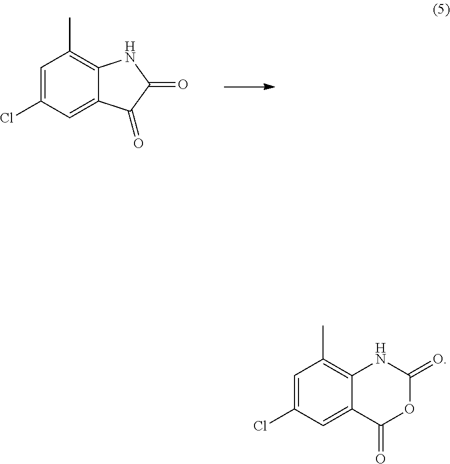

S500 specifically includes: the oxidation agent is added dropwise to a solution of the 5-chloro-7-methylisatin under stirring, and the oxidation reaction is performed at a fifth preset reaction temperature until the oxidation reaction is completed, to obtain a fifth reaction system; and the fifth reaction system is subjected to cooling, suction filtration, water-washing, and oven-drying to obtain the 5-chloro-7-methylisatoic anhydride.

In some embodiments, the oxidation agent is at least one selected from the group consisting of hydrogen peroxide, peroxyacetic acid, n-butanol peroxide, chromium trioxide, potassium permanganate, sodium hypochlorite, potassium chlorate, potassium peroxymonosulfate, and sodium persulfate.

In some embodiments, a molar ratio of the 5-chloro-7-methylisatin to the oxidation agent is in a range of 1:1.1 to 1:2.1. In some embodiments, the molar ratio of the 5-chloro-7-methylisatin to the oxidation agent is 1:1.2, 1:1.3, 1:1.4, 1:1.5, 1:1.6, 1:1.7, 1:1.8, 1:1.9, or 1:2.0.

In some embodiments, the fifth preset reaction temperature is in a range of −10° C. to 110° C. In some embodiments, the fifth preset reaction temperature is −10° C., 0° C., 10° C., 20° C., 30° C., 40° C., 50° C., 60° C., 70° C., 80° C., 90° C., or 100° C.

Specifically, a specified amount of acetic acid is fed into a reactor, and the 5-chloro-7-methylisatin obtained in the previous step is fed into the reactor under stirring; the oxidation reaction is performed at the fifth preset reaction temperature until the oxidation reaction is completed, to obtain the fifth reaction system; and the fifth reaction system is cooled and centrifuged, and a resulting filter cake is washed with water and oven-dried to obtain the target product 5-chloro-7-methylisatoic anhydride.

S600: the 5-chloro-7-methylisatoic anhydride is subjected to ammonolysis reaction with monomethylamine to obtain the 2-amino-5-chloro-N,3-dimethylbenzamide, as shown in the following equation:

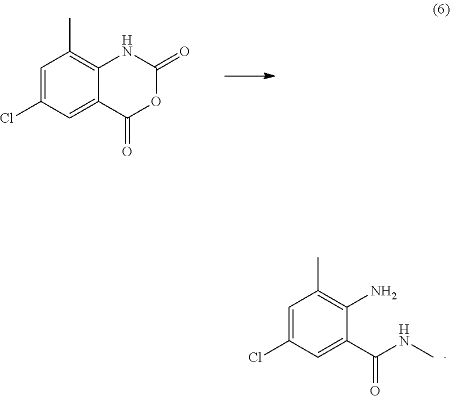

S600 specifically includes: an aqueous solution of the monomethylamine is added dropwise to a solution of the 5-chloro-7-methylisatoic anhydride under stirring, and the ammonolysis reaction is performed at a sixth preset reaction temperature until the ammonolysis reaction is completed, to obtain a sixth reaction system; and the sixth reaction system is subjected to suction filtration, water-washing, and oven-drying to obtain the 2-amino-5-chloro-N,3-dimethylbenzamide.

In some embodiments, a concentration of the aqueous solution of the monomethylamine is in a range of 10 wt % to 40 wt %. In some embodiments, the concentration of the aqueous solution of the monomethylamine is 10 wt %, 20 wt %, or 30 wt %.

In some embodiments, a solvent in the solution of the 5-chloro-7-methylisatoic anhydride is at least one selected from the group consisting of methanol, ethanol, dichloromethane, dichloroethane, acetonitrile, ethyl acetate, and water.

In some embodiments, a molar ratio of the 5-chloro-7-methylisatoic anhydride to the monomethylamine is in a range of 1:1.1 to 1:3.1. In some embodiments, the molar ratio of the 5-chloro-7-methylisatoic anhydride to the monomethylamine is 1:1.1, 1:1.2, 1:1.3, 1:1.4, 1:1.5, 1:1.6, 1:1.7, 1:1.8, 1:1.9, 1:2.0, 1:2.1, 1:2.2, 1:2.3, 1:2.4, 1:2.5, 1:2.6, 1:2.7, 1:2.8, 1:2.9, or 1:3.0.

In some embodiments, the sixth preset reaction temperature is in a range of −10° C. to 110° C. In some embodiments, the sixth preset reaction temperature is −10° C., 0° C., 10° C., 20° C., 30° C., 40° C., 50° C., 60° C., 70° C., 80° C., 90° C., or 100° C.

Specifically, a specified amount of the solvent is fed into a reactor, and under stirring, the 5-chloro-7-methylisatoic anhydride obtained in the previous step is fed into the reactor and then the aqueous solution of the monomethylamine is added dropwise; the ammonolysis reaction is performed at the sixth preset reaction temperature until the ammonolysis reaction is completed, to obtain the sixth reaction system; and the sixth reaction system is cooled and centrifuged, and a resulting filter cake is washed with water and oven-dried to obtain the target product 2-amino-5-chloro-N,3-dimethylbenzamide.

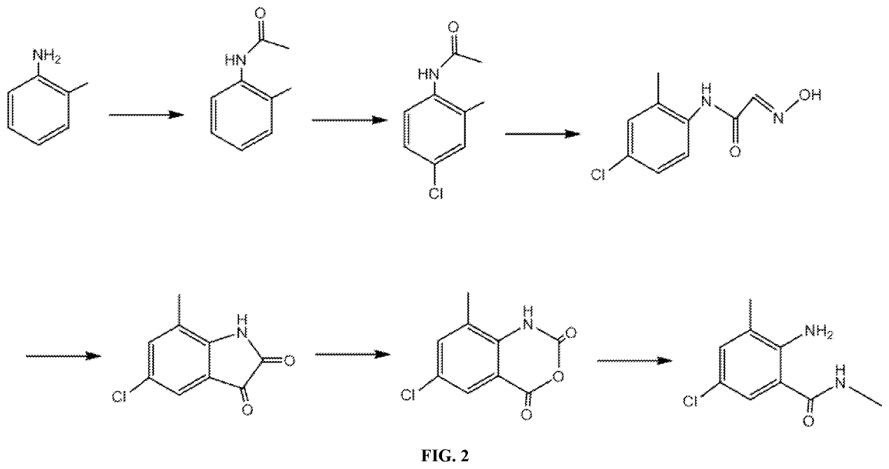

Further, a specific synthesis process of 2-amino-5-chloro-N,3-dimethylbenzamide in the present disclosure is shown in FIG. 2.

The present disclosure is described in further detail below in conjunction with examples, but the present disclosure is not limited to the examples.

### Example 1

A specific embodiment of preparation of 2-amino-5-chloro-N,3-dimethylbenzamide was provided in this example.

A basic process in this example was the specific technical implementation solution of the present disclosure described above.

Some specific details in this example were as follows:

107 kg of o-toluidine was added to a 500 mL four-neck bottle, then 210 g of acetic acid was added, stirring was started, and a first reaction was conducted at a temperature of 100° C. to 105° C. for 5 h to obtain a first reaction system. The first reaction system was cooled to a temperature of 10° C. to 15° C. and subjected to suction filtration to obtain a first filter cake. The first filter cake was washed with water until neutral and then oven-dried to obtain 141.5 g of o-methylacetanilide with a purity of 99.1% and a yield of 94.1%.

250 mL of chlorobenzene was added to a 500 mL four-neck bottle, and then 45.0 g of the o-methylacetanilide obtained in the previous step was added; stirring was started, a temperature was controlled at 35° C. to 45° C., and chlorine gas was slowly introduced, and a second reaction was performed for 4 h to obtain a second reaction system. The second reaction system was cooled to a temperature of 5° C. to 10° C. and subjected to suction filtration to obtain a second filter cake. The second filter cake was oven-dried to obtain 54.0 g of 4-chloro-2-methylacetanilide with a purity of 98.1% and a yield of 96.1%.

200 ml of water was added to a 500 mL four-neck bottle, and 37.5 g of the 4-chloro-2-methylacetanilide obtained in the previous step was added; stirring was started, a temperature was controlled at 5° C. to 10° C., and methyl nitrite gas was introduced and a third reaction was performed for 5 h to obtain a third reaction system. The third reaction system was subjected to suction filtration to obtain a third filter cake. The third filter cake was washed with water and oven-dried to obtain 39.3 g of 4-chloro-2-(hydroxyimino)-N-(2-methylphenyl) acetamide with a purity of 95.1% and a yield of 88.0%.

170 mL of an aqueous solution of sulfuric acid (98 wt %) was added to a 500 ml four-neck bottle, stirring the was started, 22.4 g of 4-chloro-2-(hydroxyimino)-N-(2-methylphenyl) acetamide obtained in the previous step was added, and a fourth reaction was conducted at a temperature of 65° C. to 70° C. for 7 h to obtain a fourth reaction system. The fourth reaction system was cooled to room temperature, slowly added to a 500 mL four-neck bottle filled with 200 mL of ice water and cooled to a temperature of 15° C. to 20° C., and then subjected to suction filtration to obtain a fourth filter cake. The fourth filter cake was oven-dried to obtain 18.0 g of 5-chloro-7-methylisatin with a purity of 95.3% and a yield of 92.0%.

130 mL of acetic acid was added to a 500 mL four-neck bottle, and stirring was started; 18.0 g of the 5-chloro-7-methylisatin obtained in the previous step was added, a temperature was controlled at 50° C. to 55° C., 11.9 g of 30 wt % hydrogen peroxide was slowly added dropwise within 1 h, and a fifth reaction was conducted for 11 h at the above temperature to obtain a fifth reaction system. The fifth reaction system was cooled to a temperature of 10° C. to 15° C. and subjected to suction filtration to obtain a fifth filter cake, and the fifth filter cake was washed with water and oven-dried to obtain 17.9 g of 5-chloro-7-methylisatoic anhydride with a purity of 96.1% and a yield of 93.0%.

200 mL of acetonitrile was added to a 500 mL four-neck bottle, and stirring was started; 17.9 g of the 5-chloro-7-methylisatoic anhydride obtained in the previous step was added, a temperature was controlled at 10° C. to 15° C., 9.3 g of a 30 wt % monomethylamine aqueous solution was added dropwise within 1 h, and a sixth reaction was then continuously conducted for 2 h to obtain a sixth reaction system. The sixth reaction system was subjected to suction filtration to obtain a sixth filter cake. The sixth filter cake was washed with water and oven-dried to obtain 15.5 g of a crude product 2-amino-5-chloro-N,3-dimethylbenzamide with a purity of 94.0% and a yield of 90%. The crude product was subjected to recrystallization with 50 mL of ethyl acetate to obtain 14.1 g of 2-amino-5-chloro-N,3-dimethylbenzamide with a purity of 98.3%.

### Example 2

A specific embodiment of preparation of 2-amino-5-chloro-N,3-dimethylbenzamide was provided in this example.

A basic process in this example was the specific technical implementation solution of the present disclosure described above.

Some specific details in this example were as follows:

214.1 kg of o-toluidine was added to a 1,000 mL four-neck bottle, then 200 g of acetic acid was added, 241.0 g of acetic anhydride was added, stirring was started, and a first reaction was conducted at a temperature of 40° C. to 45° C. for 3 h to obtain a first reaction system. The first reaction system was cooled to a temperature of 10° C. to 15° C. and subjected to suction filtration to obtain a first filter cake. The first filter cake was washed with water until neutral and then oven-dried to obtain 285.5 g of o-methylacetanilide with a purity of 98.8% and a yield of 94.4%.

250 mL of dichloroethane was added to a 500 mL four-neck bottle, and then 30.2 g of the o-methylacetanilide obtained in the previous step was added; stirring was started, a temperature was controlled at 35° C. to 45° C., and 32.0 g of NCS was added, and a second reaction was performed for 2 h to obtain a second reaction system. The second reaction system was cooled to a temperature of 5° C. to 10° C. and subjected to suction filtration to obtain a second filter cake. The second filter cake was oven-dried to obtain 34.7 g of 4-chloro-2-methylacetanilide with a purity of 98.7% and a yield of 93.2%.

200 mL of water was added to a 500 mL four-neck bottle, and 34.7 g of the 4-chloro-2-methylacetanilide obtained in the previous step was added; stirring was started, a temperature was controlled at 5° C. to 10° C., and 16.8 g of ethyl nitrite was added and a third reaction was performed for 7 h to obtain a third reaction system. The third reaction system was subjected to suction filtration to obtain a third filter cake. The third filter cake was washed with water and oven-dried to obtain 41.2 g of 4-chloro-2-(hydroxyimino)-N-(2-methylphenyl) acetamide with a purity of 96.2% and a yield of 86.6%.

300 mL of an aqueous solution of sulfuric acid (98 wt %) was added to a 500 mL four-neck bottle, stirring was started, 41.2 g of the 4-chloro-2-(hydroxyimino)-N-(2-methylphenyl) acetamide obtained in the previous step was added, and a fourth reaction was conducted at a temperature of 85° C. to 90° C. for 4 h to obtain a fourth reaction system. The fourth reaction system was cooled to room temperature, slowly added to a 100 mL four-neck bottle filled with 200 mL of ice water and cooled to a temperature of 5° C. to 10° C., and then subjected to suction filtration to obtain a fourth filter cake. The fourth filter cake was oven-dried to obtain 34.6 g of 5-chloro-7-methylisatin with a purity of 96.1% and a yield of 91.2%.

250 mL of n-butanol peroxide was added to a 500 mL four-neck bottle, and stirring was started; 34.6 g of the 5-chloro-7-methylisatin obtained in the previous step was added, a temperature was controlled at 80° C. to 85° C., and a fifth reaction was conducted for 8 h to obtain a fifth reaction system. The fifth reaction system was cooled to a temperature of 10° C. to 15° C. and subjected to suction filtration to obtain a fifth filter cake, and the fifth filter cake was washed with water and oven-dried to obtain 34.8 g of 5-chloro-7-methylisatoic anhydride with a purity of 97.2% and a yield of 94.1%.

300 mL of ethyl acetate was added to a 500 mL four-neck bottle, and stirring was started; 34.8 g of the 5-chloro-7-methylisatoic anhydride obtained in the previous step was added, a temperature was controlled at 20° C. to 315° C., 18.9 g of a 30 wt % monomethylamine aqueous solution was added dropwise within 2 h, and a sixth reaction was then continuously conducted for 3 h to obtain a sixth reaction system. The sixth reaction system was subjected to suction filtration to obtain a sixth filter cake. The sixth filter cake was washed with water and oven-dried to obtain 31.3 g of a crude product 2-amino-5-chloro-N,3-dimethylbenzamide with a purity of 93.3% and a yield of 92.1%. The crude product was subjected to recrystallization with 100 mL of 95% ethanol aqueous solution to obtain 30.2 g of 2-amino-5-chloro-N,3-dimethylbenzamide with a purity of 98.8%.

There are currently the following two major methods for synthesizing 2-amino-5-chloro-N,3-dimethylbenzamide. A first method: o-toluidine as a raw material is subjected to condensation with trichloroacetaldehyde hydrate and hydroxylamine hydrochloride to obtain 2-(hydroxyimino)-N-(2-methylphenyl) acetamide, then the 2-(hydroxyimino)-N-(2-methylphenyl) acetamide is subjected to cyclization in the presence of concentrated sulfuric acid to obtain 7-methylisatin, the 7-methylisatin is oxidized by hydrogen peroxide to obtain 7-methylisatoic anhydride, the 7-methylisatoic anhydride is subjected to ring-opening with methylamine gas to obtain 2-amino-N,3-dimethylbenzamide, and finally the 2-amino-N,3-dimethylbenzamide is chlorinated to obtain 2-amino-5-chloro-N,3-dimethylbenzamide. A specific route is as follows:

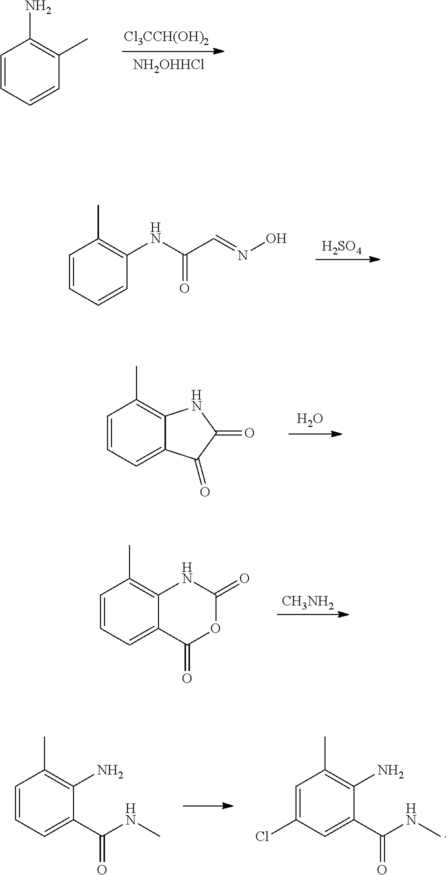

This route has a low raw material cost, but involves a large amount of sodium sulfate during a reaction and leads to large amounts of gas, water, and residue wastes.

A second method: m-toluic acid is nitrified with a mixed acid (a mixture of concentrated nitric acid and concentrated sulfuric acid) to obtain 2-nitro-3 methylbenzoic acid, and then the 2-nitro-3 methylbenzoic acid is hydrogenated at a high temperature and a high pressure to reduce nitro into amino, then chlorinated by NCS, and then reacted with monomethylamine to obtain 2-amino-5-chloro-N,3-dimethylbenzamide. A specific route is as follows:

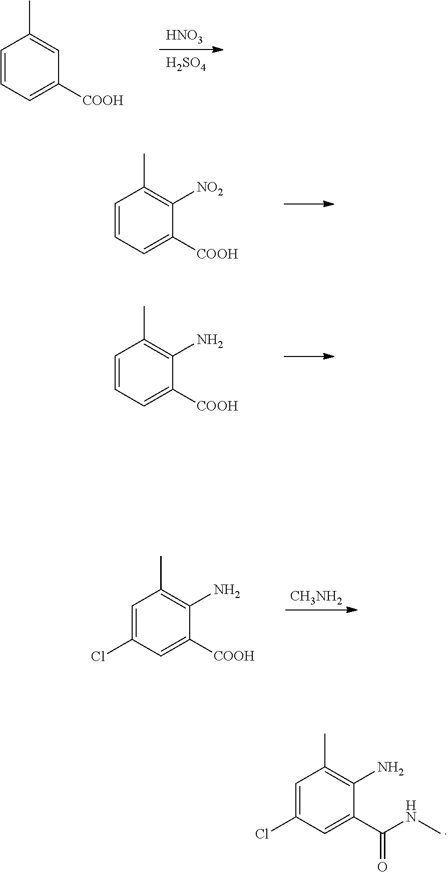

This route is short, but involves nitrification and high-pressure hydrogenation. Due to multiple active sites during nitrification, a target nitrification product has a selectivity of less than 40%. A separation process of various nitrification products is complicated, and by-products of the separation process are mostly solid wastes. In addition, the hydrogenation involves a high temperature and a high pressure, which increases unsafe factors in the production process.

Compared with the methods in the prior art, in the method of the present disclosure, the reaction temperature is controlled at 135° C. or lower, high-pressure hydrogenation is avoided, and the target product 2-amino-5-chloro-N,3-dimethylbenzamide can have a purity of 93.3% and a yield of 92.1%, that is, the product has a high purity and a high yield. In addition, the method of the present disclosure involves mild reaction conditions and simple and safe operations, results in small amounts of gas, water, and residue wastes, has prominent social and economic benefits, and is suitable for large-scale industrial production.

The above are merely preferred embodiments of the present disclosure, and the scope of the present disclosure is not limited thereto. Any equivalent structural change made using the content of the specification and the accompanying drawings of the present disclosure under the inventive concept of the present disclosure, or direct/indirect application thereof in other related technical fields, shall fall within the scope of the present disclosure.

What is claimed is:

1. A method for preparing 2-amino-5-chloro-N,3-dimethylbenzamide, comprising the following steps:
subjecting o-toluidine to acylation reaction with an acylation agent to obtain o-methylacetanilide, as shown in the following equation: 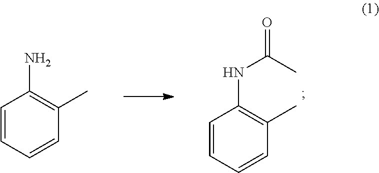
subjecting the o-methylacetanilide to chlorination reaction with a chlorination agent to obtain 4-chloro-2-methylacetanilide, as shown in the following equation: 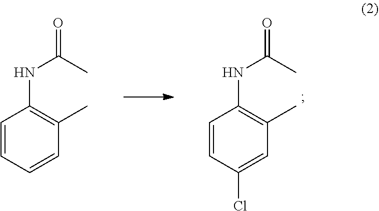
subjecting the 4-chloro-2-methylacetanilide to oximation reaction with an oximation agent to obtain 4-chloro-2-(hydroxyimino)-N-(2-methylphenyl) acetamide, as shown in the following equation: 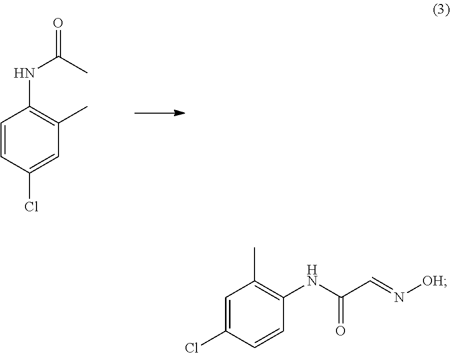
subjecting the 4-chloro-2-(hydroxyimino)-N-(2-methylphenyl) acetamide to cyclization reaction with sulfuric acid to obtain 5-chloro-7-methylisatin, as shown in the following equation: 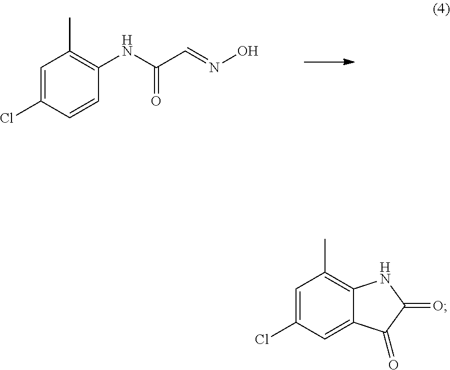
subjecting the 5-chloro-7-methylisatin to oxidation reaction with an oxidation agent to obtain 5-chloro-7-methylisatoic anhydride, as shown in the following equation: 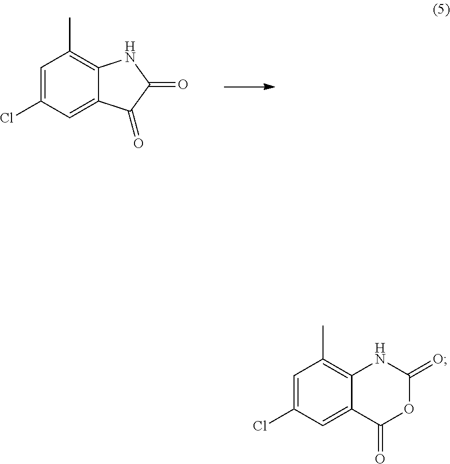
and
subjecting the 5-chloro-7-methylisatoic anhydride to ammonolysis reaction with monomethylamine to obtain the 2-amino-5-chloro-N,3-dimethylbenzamide, as shown in the following equation:

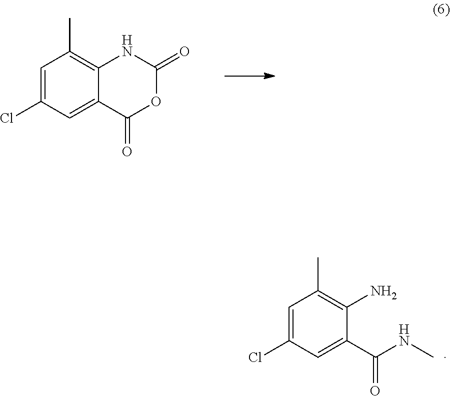

2. The method of claim 1, wherein the subjecting o-toluidine to acylation reaction with an acylation agent to obtain o-methylacetanilide comprises:
adding the acylation agent to a solution of the o-toluidine under stirring, and performing the acylation reaction at a first preset reaction temperature until the acylation reaction is completed, to obtain a first reaction system; and subjecting the first reaction system to cooling, suction filtration, water-washing, and oven-drying to obtain the o-methylacetanilide,
wherein the solution of the o-toluidine comprises at least one solvent selected from the group consisting of dichloromethane, dichloroethane, acetic acid, acetic anhydride, chlorobenzene, and xylene; the acylation agent is acetic acid or acetic anhydride; a molar ratio of the o-toluidine to the acylation agent is in a range of 1:0.9 to 1:1.5; a molar ratio of the o-toluidine to the solvent in the solution of the o-toluidine is in a range of 1:3 to 1:7; and the first preset reaction temperature is in a range of −10° C. to 135° C.

3. The method of claim 1, wherein the subjecting the o-methylacetanilide to chlorination reaction with a chlorination agent to obtain 4-chloro-2-methylacetanilide comprises:
adding the chlorination agent to a solution of the o-methylacetanilide under stirring, and performing the chlorination reaction at a second preset reaction temperature until the chlorination reaction is completed, to obtain a second reaction system; and subjecting the second reaction system to cooling, suction filtration, and oven-drying to obtain the 4-chloro-2-methylacetanilide,
wherein a solvent in the solution of the o-methylacetanilide is at least one selected from the group consisting of methanol, ethanol, dichloromethane, dichloroethane, acetic acid, chlorobenzene, and water; the chlorination agent is at least one selected from the group consisting of chlorine gas, N-chlorosuccinimide (NCS), hydrogen peroxide/hydrochloric acid, oxygen gas/hydrochloric acid, sulfuryl chloride, thionyl chloride, phosphorus trichloride, phosphorus pentachloride, and sodium hypochlorite; a molar ratio of the o-methylacetanilide to the chlorination agent is in a range of 1:1.1 to 1:1.5; a molar ratio of the o-methylacetanilide to the solvent in the solution of the o-methylacetanilide is in a range of 1:3 to 1:8; and the second preset reaction temperature is in a range of −10° C. to 105° C.

4. The method of claim 1, wherein the subjecting the 4-chloro-2-methylacetanilide to oximation reaction with an oximation agent to obtain 4-chloro-2-(hydroxyimino)-N-(2-methylphenyl) acetamide comprises:
adding the oximation agent to a solution of the 4-chloro-2-methylacetanilide under stirring, and performing the oximation reaction at a third preset reaction temperature until the oximation reaction is completed, to obtain a third reaction system; and subjecting the third reaction system to suction filtration, water-washing, and oven-drying to obtain the 4-chloro-2-(hydroxyimino)-N-(2-methylphenyl) acetamide,
wherein a solvent in the solution of the 4-chloro-2-methylacetanilide is selected from the group consisting of methanol, ethanol, dichloromethane, dichloroethane, acetic acid, chlorobenzene, and water; the oximation agent is selected from the group consisting of methyl nitrite, ethyl nitrite, n-butyl nitrite, isopropyl nitrite, isoamyl nitrite, and n-amyl nitrite; a molar ratio of the 4-chloro-2-methylacetanilide to the oximation agent is in a range of 1:1.1 to 1:2.0; a molar ratio of the 4-chloro-2-methylacetanilide to the solvent in the solution of the 4-chloro-2-methylacetanilide is in a range of 1:3 to 1:20; and the third preset reaction temperature is in a range of −10° C. to 115° C.

5. The method of claim 1, wherein the subjecting the 4-chloro-2-(hydroxyimino)-N-(2-methylphenyl) acetamide to cyclization reaction with sulfuric acid to obtain 5-chloro-7-methylisatin comprises:
adding the 4-chloro-2-(hydroxyimino)-N-(2-methylphenyl) acetamide to an aqueous solution of the sulfuric acid under stirring, and performing the cyclization reaction at a fourth preset reaction temperature until the cyclization reaction is completed, to obtain a fourth reaction system; and subjecting the fourth reaction system to cooling, suction filtration, and oven-drying to obtain the 5-chloro-7-methylisatin,
wherein a concentration of the aqueous solution of the sulfuric acid is 75 wt % to 98 wt %; a molar ratio of the 4-chloro-2-(hydroxyimino)-N-(2-methylphenyl) acetamide to the sulfuric acid is in a range of 1:3 to 1:30; and the fourth preset reaction temperature is in a range of 20° C. to 115° C.

6. The method of claim 1, wherein the subjecting the 5-chloro-7-methylisatin to oxidation reaction with an oxidation agent to obtain 5-chloro-7-methylisatoic anhydride comprises:
adding the oxidation agent dropwise to a solution of the 5-chloro-7-methylisatin under stirring, and performing the oxidation reaction at a fifth preset reaction temperature until the oxidation reaction is completed, to obtain the fifth reaction system; and subjecting the fifth reaction system to cooling, suction filtration, water-washing, and oven-drying to obtain the 5-chloro-7-methylisatoic anhydride,
wherein the oxidation agent is at least one selected from the group consisting of hydrogen peroxide, peroxyacetic acid, n-butanol peroxide, chromium trioxide, potassium permanganate, sodium hypochlorite, potassium chlorate, potassium peroxymonosulfate, and sodium persulfate; a molar ratio of the 5-chloro-7-methylisatin to the oxidation agent is in a range of 1:1.1 to 1:2.1; and the fifth preset reaction temperature is in a range of −10° C. to 110° C.

7. The method of claim 1, wherein the subjecting the 5-chloro-7-methylisatoic anhydride to ammonolysis reaction with monomethylamine to obtain the 2-amino-5-chloro-N,3-dimethylbenzamide comprises:
adding an aqueous solution of the monomethylamine dropwise to a solution of the 5-chloro-7-methylisatoic anhydride under stirring, and performing the ammonolysis reaction at a sixth preset reaction temperature until the ammonolysis reaction is completed, to obtain the sixth reaction system; and subjecting the sixth reaction system to suction filtration, water-washing, and oven-drying to obtain the 2-amino-5-chloro-N,3-dimethylbenzamide,
wherein a concentration of the aqueous solution of the monomethylamine is in a range of 10 wt % to 40 wt %; a solvent in the solution of the 5-chloro-7-methylisatoic anhydride is at least one selected from the group consisting of methanol, ethanol, dichloromethane, dichloroethane, acetonitrile, ethyl acetate, and water; a molar ratio of the 5-chloro-7-methylisatoic anhydride to the monomethylamine is in a range of 1:1.1 to 1:3.1; and the sixth preset reaction temperature is in a range of −10° C. to 110° C.
